(19) United States
(12) Patent Application Publication
(10) Pub. No.: US 2022/0160981 A1
(43) Pub. Date: May 26, 2022

(54) System and Method For Inserting A Medical Device

(71) Applicant: New York University, New York, NY (US)

(72) Inventors: Luis Fernando Angel, Hartsdale, NY (US); Gabriella Cammarata, Brooklyn, NY (US)

(21) Appl. No.: 17/533,369

(22) Filed: Nov. 23, 2021

Related U.S. Application Data
(60) Provisional application No. 63/117,729, filed on Nov. 24, 2020.

(51) Int. Cl. A61M 16/04

(52) U.S. Cl. CPC A61M 16/0495; A61M 16/0493

(57) ABSTRACT

Embodiments of the present disclosure relate to an auxiliary device and medical equipment for inserting a medical device. Embodiments of the disclosure comprise a bite part connected to a proximal end of a guiding tube. The bite part comprises a body, a first retaining part and a second retaining part, wherein the body has with a first toothing part, a first connecting part, a second toothing part and a second connecting part, and an insertion cavity in communication with the guiding tube. The first toothing part and the second toothing part occlude the upper and lower teeth, and the first retaining part and the second retaining part are respectively disposed at the first toothing part and the second toothing part, thereby forming an avoiding space for endotracheal intubation at the side of the first connecting part and/or the second connecting part.

### CROSS-REFERENCE TO RELATED APPLICATIONS

The present application claims priority to U.S. Provisional Application No. 63/117,729, filed on Nov. 24, 2020, the contents of which are incorporated herein by reference in their entirety for all purposes.

### FIELD

The present disclosure relates to the technical field of medical devices, and specifically to an auxiliary device and medical equipment for inserting an interventional medical device.

### BACKGROUND

In a surgery, such as throat surgery or tracheotomy, endotracheal intubation is often adopted to assist breathing. Endotracheal intubation is a method for inserting an endotracheal tube (ETT) into the trachea or bronchus via the oral or nasal cavity and the glottis, to provide the best conditions for ventilation or oxygen supply.

Meanwhile, in order to ensure the intubation effect of the ETT, a bronchoscope is usually inserted into the ETT during endotracheal intubation, and thus the position of the ETT can be obtained and the effect of the intubation can be checked through the bronchoscope. When the surgery for patients suffering from infectious respiratory diseases, such as COVID-19, is performed, the channel for ETT is in an open state and lots of secretion aerosols will be ejected, which poses a high risk of infection for medical staff. Furthermore, during conventional endotracheal intubation, it is necessary to connect a respirator and adjust the parameters after the placement of the ETT to meet the ventilation requirements. This respirator requires frequent multiple-person operation. When performing a surgery on patients suffering from infectious respiratory diseases such as COVID-19, if there is fewer medical staff in the operation room, it will increase the difficulty in operations, and if the number of medical staff is increased, the medical staff will be exposed to higher risk of infection.

### SUMMARY

An object of the present disclosure is to provide an auxiliary device for inserting the interventional medical device, which can improve the technical problems of difficult operation and high risk of infection in the prior art.

Another object of the present disclosure is to provide a medical device, which can improve the technical problems of difficult operation and high risk of infection in the prior art.

The embodiments of the present disclosure can be implemented as follows:

The embodiments of the present disclosure relate to an auxiliary device for inserting the interventional medical device, which comprises a bite part and a guiding tube, and the bite part is connected to the proximal end of the guiding tube; the bite part comprises a body, a first retaining part and a second retaining part, wherein the body is provided with a first toothing part, a first connecting part, a second toothing part and a second connecting part which are connected end to end; the first toothing part, the first connecting part, the second toothing part and the second connecting part form an insertion cavity in communication with the guiding tube, thereby forming a passage inside the bite part and the guiding tube for the penetration of the interventional medical device; the first toothing part and the second toothing part are respectively used for occluding the upper and lower teeth of the human body; the first retaining part and the second retaining part are respectively disposed at the first toothing part and the second toothing part, thereby forming an avoiding space for endotracheal intubation at the side of the first connecting part and/or said the second connecting part away from the insertion cavity.

Alternatively, the first connecting part and/or the second connecting part are/is provided with a groove, wherein the groove extends in the direction from the proximal end to the distal end of the body, and the groove is used to form the avoiding space.

Alternatively, the groove is formed by recessing the outer wall of the first connecting part and/or the second connecting part towards the insertion cavity, and the two ends of the groove penetrate the proximal end and the distal end of the bite part, respectively.

Alternatively, the insertion auxiliary device is also comprised of a tongue depressor, which is fixedly connected to the distal end of the guiding tube.

Alternatively, the guiding tube is provided with an inside curve and an outside curve that are opposite to each other, and the tongue depressor is fixedly connected to the distal end of the inside curve.

Alternatively, the tongue depressor is comprised of a tongue depressing part and a support part that are interconnected, wherein the tongue depressing part is fixedly connected to the distal end of the guiding tube, and the support part is slantways disposed opposite to the tongue depressing part, thereby forming an accommodation space for the interventional medical device between the tongue depressing part and the support part.

Alternatively, the first retaining part is disposed at the edge of the proximal end of the first toothing part, and the second retaining part is disposed at the edge of the proximal end of the second toothing part.

Alternatively, the insertion auxiliary device is also provided with an oxygen passage, and the oxygen passage extends from the proximal end of the bite part to the distal end face of the guiding tube.

Alternatively, the oxygen passage is arranged outside the bite part and the guiding tube.

The embodiments of the present disclosure relate to a medical device, comprising an auxiliary device for inserting the interventional medical device, which comprises a bite part and a guiding tube, and the bite part is connected to the proximal end of the guiding tube; the bite part comprises a body, a first retaining part and a second retaining part, wherein the body is provided with a first toothing part, a first connecting part, a second toothing part and a second connecting part which are connected end to end; the first toothing part, the first connecting part, the second toothing part and the second connecting part form an insertion cavity in communication with the guiding tube, thereby forming a passage inside the bite part and the guiding tube for the penetration of the interventional medical device; the first toothing part and the second toothing part are respectively used for occluding the upper and lower teeth of the human body; the first retaining part and the second retaining part are respectively disposed at the first toothing part and the second toothing part, thereby forming an avoiding space for endotracheal intubation at the side of the first connecting part and/or said the second connecting part away from the insertion cavity.

The beneficial effects of the auxiliary device and medical equipment for inserting the interventional medical device in the embodiments of the present disclosure include, for instance:

The embodiments of the present disclosure relate to an auxiliary device for inserting the interventional medical device, which comprises a bite part and a guiding tube, and the bite part is connected to the proximal end of the guiding tube. The bite part comprises a body, a first retaining part and a second retaining part, wherein the body is provided with a first toothing part, a first connecting part, a second toothing part and a second connecting part which are connected end to end and form an insertion cavity communicated with the guiding tube, thereby forming a passage inside the bite part and the guiding tube for the penetration of the interventional medical device. The first toothing part and the second toothing part are respectively used for occluding the upper and lower teeth of the human body, and the first retaining part and the second retaining part are respectively disposed at the first toothing part and the second toothing part, thereby forming an avoiding space for endotracheal intubation at the side of the first connecting part and/or the second connecting part away from the insertion cavity. In use, the patient bites the body of the bite part, and the distal end of the guiding tube extends into the trachea of the patient. The upper and lower teeth of the patient bite the first toothing part and the second toothing part respectively, and the first retaining part and the second retaining part respectively abut against the upper and lower lips of patient to limit the insertion auxiliary device. The patient's mouth is in an open state, and the avoiding space is connected with the patient's mouth, so that the ETT can enter into the mouth and extend into the human trachea. In this way, the interventional medical device and the ETT are separated by inserting the auxiliary device, which can effectively avoid the influence of the interventional medical device on the ventilation of ETT, thus reducing the difficulty in operation and the risk of infection. At the same time, the whole procedure can be performed directly under the interventional medical device, which is helpful to reduce the difficulty of operation and shorten the operation time. Furthermore, the interventional medical device is seated in the passage for inserting the auxiliary device, which prevents the insertion and removal of the interventional medical device from carrying the patient's secretions, and further reduces the risk of infection.

The embodiments of the present disclosure also relate to a medical device, which comprises the auxiliary device for inserting the interventional medical device. As the medical device comprises the auxiliary device for inserting the interventional medical device, the medical device can also bring the beneficial effects of reducing the difficulty in operation, lowering the risk of infection, and shortening the operation time.

### BRIEF DESCRIPTION OF THE DRAWINGS

A brief description of the drawings is given below. It should be understood that the following drawings are merely illustrative of certain embodiments of the present disclosure and are not intended to limit the present disclosure. For those of ordinary skill in the art, without creative work, other related drawings can be obtained from these drawings.

FIG. 1 schematically illustrates the structure of the auxiliary device provided by the embodiments of the present disclosure from the first perspective;

FIG. 2 schematically illustrates the structure of the bite part of the insertion auxiliary device provided by the embodiments of the present disclosure;

FIG. 3 schematically illustrates the structure of the insertion auxiliary device provided by the embodiments of the present disclosure from the second perspective;

FIG. 4 schematically illustrates the structure of another kind of bite part of the insertion auxiliary device provided by the embodiments of the present disclosure;

FIG. 5 schematically illustrates the partial structure of the insertion auxiliary device provided by the embodiments of the present disclosure.

Legend: 100—insertion auxiliary device; 110—bite part; 111—body; 11110—the first toothing part; 1112—the first connecting part; 1113—the second toothing part; 1114—the second connecting part; 1115—groove; 112—the first retaining part; 113—the second retaining part; 114—the first extending part; 115—the second extending part; 116—avoiding space; 117—insertion cavity; 120—guiding tube; 121—inside curve; 122—outside curve; 130—tongue depressor; 131—tongue depressing part; 132—support part; 133—accommodation space; 140—oxygen passage; 141—oxygen inlet.

### DETAILED DESCRIPTION

In order to make the objects, technical proposals and advantages of the present disclosure more comprehensible, the technical proposals in the embodiments of the present disclosure will be further described in detail below with reference to the drawings. Obviously, the described embodiments are part of the embodiments of the present disclosure and not all of them. Generally, the components of the embodiments of the present disclosure described and shown in the drawings herein may be arranged and designed in a variety of different configurations.

The following detailed descriptions of the embodiments of the present disclosure provided in the drawings are not intended to limit the protection scope of the present disclosure for which protection is required, but to represent embodiments of the present disclosure. Based on the embodiments of the present disclosure, all other embodiments acquired by the common technicians in this field without creative work, shall be in the protection scope of the present disclosure.

Similar figures and letters indicate similar items in the following drawings; therefore, once an item is defined in one drawing, it will be unnecessary to further define and explain it in the subsequent drawings.

Terms indicating the orientation or position such as “upper”, “lower”, “inner” and “outer” appearing in the description of this disclosure refer to the orientation or position shown in the drawings, or the orientation or position of this disclosure commonly placed in use, and are merely for convenience and simplification of the description of this disclosure, instead of indicating or implying that the device or element referred to must have a specific orientation and be constructed and operated in a particular orientation; therefore, they cannot be understood as a restriction on this disclosure.

Furthermore, the terms “first”, “second” and so on are only used for differentiation in description, and cannot be understood as indication or implication of the relative importance.

The features in the embodiments of this disclosure may be combined with each other if they are not in conflict.

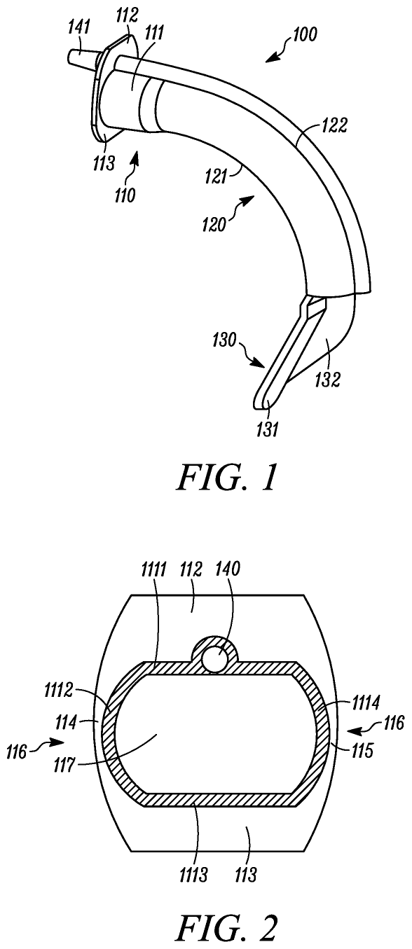

FIG. 1 schematically illustrates the structure of the insertion auxiliary device 100 provided by this embodiment of this disclosure from the first perspective; FIG. 2 schematically illustrates the structure of the bite part 110 of the insertion auxiliary device 100 provided by this embodiment of this disclosure. Please refer to FIG. 1 and FIG. 2. This embodiment relates to an insertion auxiliary device 100, which is used to assist the insertion of the interventional medical device into the trachea of the human body. The interventional medical device referred to in the description of this embodiment is a bronchoscope.

The insertion auxiliary device 100 comprises a bite part 110 and a guiding tube 120, and the bite part 110 is connected to the proximal end of the guiding tube 120. The bite part 110 comprises a body 111, a first retaining part 112 and a second retaining part 113, wherein the body 111 is provided with a first toothing part 1111, a first connecting part 1112, a second toothing part 1113 and a second connecting part 1114 which are connected end to end and form an insertion cavity 117 in communication with the guiding tube 120, thereby forming a passage for the penetration of the bronchoscope (not illustrated in the drawing) inside the bite part 110 and the guiding tube 120. The first toothing part 1111 and the second toothing part 1113 are respectively used for occluding the upper and lower teeth of the human body, and the first retaining part 112 and the second retaining part 113 are respectively disposed at the first toothing part 1111 and the second toothing part 1113, thereby forming an avoiding space 116 for inserting the duct of the air supply duct (not illustrated in the drawing) at the side of the first connecting part 1112 and/or the second connecting part 1114 away from the insertion cavity 117.

In use, the patient bites the body 111 of the bite part 110. The upper and lower teeth of the patient bite the first toothing part 1111 and the second toothing part 1113 respectively, and the first retaining part 112 and the second retaining part 113 respectively abut against the upper and lower lips of the patient to limit the insertion auxiliary device 100; meanwhile, the distal end of the guiding tube 120 extends into the trachea of the patient to guide the bronchoscope into the trachea, so that the medical staff can insert the ETT into the trachea directly under the bronchoscope. The patient's mouth is in an open state, and the avoiding space 116 is in communication with the patient's mouth, so that the ETT can enter the mouth through the avoiding space 116 and extend into the trachea. In this way, the bronchoscope and the ETT are separated by the insertion auxiliary device 100, which can effectively avoid the influence of the bronchoscope on the ventilation of ETT, thus reducing the difficulty in operation and the risk of infection.

In the description of this embodiment, the “proximal end” refers to the end adjacent to the outside of the human body during use, and correspondingly, the “distal end” refers to the end adjacent to the inside of the human body during use. For instance, for the insertion auxiliary device 100 provided by this embodiment, during use, the bite part 110 is arranged at the patient's mouth, and the guiding tube 120 extends to the trachea of the human body, therefore, the bite part 110 is arranged at the proximal end of the insertion auxiliary device 100, and the guiding tube 120 is arranged at the distal end of the insertion auxiliary device 100. Similarly, the end of the guiding tube 120 connected to the bite part 110 is the proximal end of the guiding tube 120, and the end of the bite part 110 connected to the guiding tube 120 is the distal end of the bite part 110.

The insertion auxiliary device 100 provided by this embodiment is further described as follows:

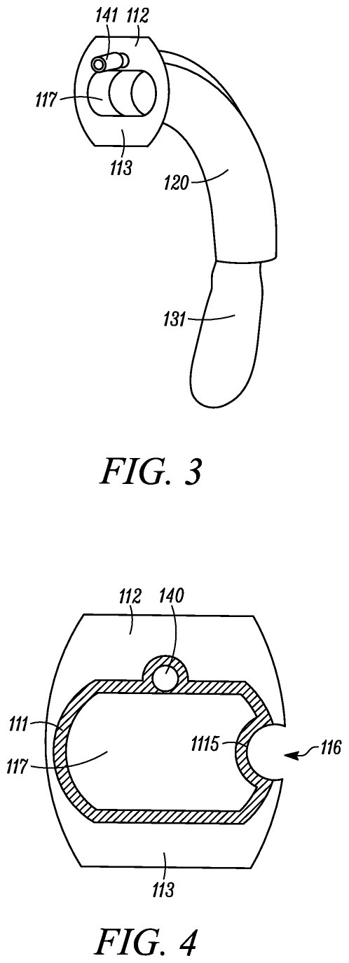

FIG. 3 schematically illustrates the structure of the insertion auxiliary device 100 provided by this embodiment from the second perspective. Please refer to FIG. 1, FIG. 2, and FIG. 3. In this embodiment, the bite part 110 comprises a body 111, a first retaining part 112 and a second retaining part 113; the first retaining part 112 and the second retaining part 113 are fixedly connected to the proximal end of the body 111 in an opposite way; in use, the patient bites the body 111 to keep the mouth opened; at the same time, the first retaining part 112 and the second retaining part 113 respectively abut against the upper and lower lips of the patient to limit the insertion auxiliary device 100. The body 111 is tubular, in which there is an insertion cavity 117 through the body 111. Specifically, the body 111 comprises a first toothing part 1111, a first connecting part 1112, a second toothing part 1113 and a second connecting part 1114 which are connected end to end and form an insertion cavity 117. The first toothing part 1111 and the second toothing part 1113 are both flat-shaped to facilitate contact with teeth, and the first connecting part 1112 and the second connecting part 1114 are arc-shaped.

The first retaining part 112 is disposed at the edge of the proximal end of the first toothing part 1111, and the second retaining part 113 is disposed at the edge of the proximal end of the second toothing part 1113. Specifically, the first retaining part 112 is the protrusion formed by the outer wall of the proximal end of the first toothing part 1111 protruding in a direction away from the insertion cavity 117; the second retaining part 113 is the protrusion formed by the outer wall of the proximal end of the second toothing part 1113 protruding in a direction away from the insertion cavity 117. Meanwhile, the end face of the first retaining part 112 away from the first toothing part 1111 is an arc convex surface, and the end face of the second retaining part 113 away from the second toothing part 1113 is an arc convex surface. The first retaining part 112 and the second retaining part 113 are respectively disposed at the first toothing part 1111 and the second toothing part 1113, so that an avoiding space 116 is formed at the side of the first connecting part 1112 and/or the second connecting part 1114 away from the insertion cavity 117. At the time of use, the patient's mouth is opened, and the ETT enters the patient's body through the space between the upper and lower teeth after passing through the avoiding space 116.

Specifically, the bite part 110 also comprises a first extending part 114 disposed at the proximal end of the first connecting part 1112 and a second extending part 115 disposed at the proximal end of the second connecting part 1114; both ends of the first extending part 114 are respectively connected with the first retaining part 112 and the second retaining part 113, and both ends of second extending part 115 are respectively connected with the first retaining part 112 and the second retaining part 113. Therefore, the first retaining part 112 and the second retaining part 113 are connected as a whole to ensure the product performance. The height of the protrusion between the first extending part 114 and the second extending part 115 should be small enough to avoid the first extending part 114 and the second extending part 115 from blocking the avoiding space 116. Specifically, the height of the protrusion between the first extending part 114 and the second extending part 115 should ensure that the space between the upper and lower teeth is visible during use.

In this embodiment, the bite part 110 is provided with both the first extending part 114 and the second extending part 115. In other embodiments, in order to ensure sufficient avoiding space 116 and better avoidance effect, only the first extending part 114 can be arranged, so that the space on the side of the second connecting part 1114 away from the insertion cavity 117 is used as the avoiding space 116, or only the first retaining part 112 and the second retaining part 113 are arranged.

FIG. 4 schematically illustrates the structure of another kind of bite part 110 of the insertion auxiliary device 100 provided by this embodiment. Please refer to FIG. 4. In this embodiment, the second connecting part 1114 is also provided with a groove 1115, wherein the groove 1115 extends along the direction from the proximal end to the distal end of the body 111, to form the avoiding space 116 through the groove 1115. Meanwhile, the second extending part 115 has a notch corresponding to the groove 1115, so that the depression penetrates through the proximal end of the bite part 110. In other embodiments, it is acceptable if only the second extending part 115 is arranged, as a result, the avoiding space 116 is arranged at the side of the first connecting part 1112 away from the insertion cavity 117. At this moment, the groove 1115 can be arranged at the first connecting part 1112 to form the avoiding space 116, which is helpful to improve the avoidance effect, or a notch can be arranged at the first extending part 114 to make the depression penetrate through the proximal end of the bite part 110. Further, the groove 1115 is formed by recessing the outer wall of the first connecting part 1112 and/or the second connecting part 1114 towards the insertion cavity 117, and the two ends of the depression penetrate through the proximal end and the distal end of the bite part 110, respectively.

With reference to FIG. 1., the guiding tube 120 is an arc-shaped elbow to adapt to the human anatomical structure and reduce the influence on the human body caused by the insertion of the guiding tube 120 into the human body. Specifically, the guiding tube 120 is provided with an inside curve 121 and an outside curve 122 that are opposite to each other. Further, the peripheral size of the body 111 of the bite part 110 is larger than that of the guiding tube 120.

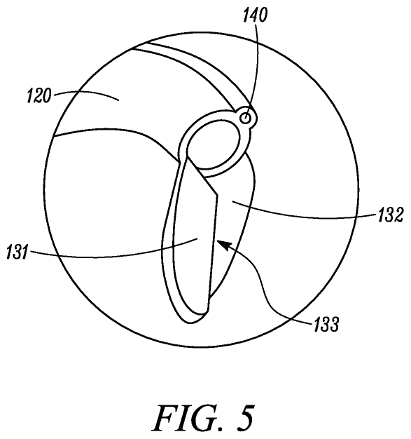

FIG. 5 schematically illustrates the partial structure of the insertion auxiliary device 100 provided by this embodiment. With reference to FIG. 1 and FIG. 5, the insertion auxiliary device 100 is also comprised of a tongue depressor 130, and the tongue depressor 130 is fixedly connected to the distal end of the guiding tube 120. Therefore, before inserting the guiding tube 120 into the patient's mouth, the tongue depressor 130 first enters the patient's mouth, and then the tongue depressor 130 presses the patient's tongue, thus making the guiding tube 120 enter the human throat more conveniently across the tongue. Moreover, when the tongue depressor 130 enters the human throat and continues to extend inward, it can guide the guiding tube 120 into the human trachea, avoiding the insertion of the guiding tube 120 into the human esophagus. Specifically, the tongue depressor 130 is fixedly connected to the distal end of the inside curve 121 of the guiding tube 120.

Further, the tongue depressor 130 is comprised of a tongue depressing part 131 and a support part 132 that are interconnected, and the tongue depressing part 131 is fixedly connected to the distal end of the guiding tube 120. When used, the outer wall of the tongue depressing part 131 is against the patient's tongue to hold the tongue and ensure that the guiding tube 120 enters the human throat. The support part 132 is fixedly connected to a side edge of the tongue depressing part 131, and the support part 132 is slantways disposed opposite to the tongue depressing part 131 to form an L-shaped tongue depressor 130, and form an accommodation space 133 between the support part 132 and the tongue depressing part 131 connected with the guiding tube 120. When the bronchoscope extends out of the inner cavity of the guiding tube 120, the distal end of the bronchoscope is arranged in the accommodation space 133, and the bronchoscope is confined in the containment space using the support part 132 and the tongue depressing part 131, thus avoiding the contact between the bronchoscope and the trachea, or the possible contamination of the bronchoscope caused by the mucus secreted by the human body. At the same time, the support part 132 can also enhance the strength of the tongue depressing part 131.

Further, the wall surface of the support part 132 away from the tongue depressing part 131 is a cambered surface, and the distance between the cambered surface and the tongue depressing part 131 gradually decreases along the direction from the proximal end to the distal end of the tongue depressing part 131. Alternatively, the tongue depressor 130, the guiding tube 120 and the bite part 110 are made in one piece. Further, the insertion auxiliary device 100 is made of transparent material.

With reference to FIG. 1, FIG. 2, and FIG. 3 the insertion auxiliary device 100 is also provided with an oxygen passage 140, and oxygen passage 140 extends from the proximal end of the bite part 110 to the distal end face of the guiding tube 120, thus supplying oxygen through the oxygen passage 140. Specifically, the first retaining part 112 is provided with an oxygen passage 141, which forms the proximal end of the oxygen passage 140 and is connected with the oxygen supply part (not illustrated in the drawing) through the oxygen passage 141 during use. The outer wall of the bite part 110 and the guiding tube 120 are provided with a cylindrical protrusion, and the oxygen passage 140 is a through-hole arranged on the cylindrical protrusion, that is, the oxygen passage 140 is arranged outside the bite part 110 and the guiding tube 120. Further, in order to avoid the cylindrical protrusion at the first toothing part 1111 from affecting the occlusion and avoiding the occlusion from affecting the ventilation of the oxygen passage 140, the cylindrical protrusion forming the oxygen passage 140 is made of elastic material, which can produce elastic deformation under the action of occlusal force, and at least part of the cross section of the through-hole forming the oxygen passage 140 is arranged on one side of the outer wall of the first toothing part 1111 adjacent to the insertion cavity 117 (as illustrated in FIG. 2).

The position of the oxygen passage 140 is not restricted and can be arranged at other positions according to the demand. For instance, the oxygen passage 140 can be arranged outside the first connecting part 1112 or outside the second connecting part 1114, provided oxygen can be supplied through the oxygen passage 140.

According to this embodiment, an auxiliary device for inserting the insertion auxiliary device 100 is provided, and the working principle of the insertion auxiliary device 100 is as follows:

In use, first insert the insertion auxiliary device 100 into the patient's body until the bite part 110 is arranged in the patient's mouth, and the upper and lower teeth of the patient are respectively occluded at the first toothing part 1111 and the second toothing part 1113. At this moment, the tongue depressor 130 of the insertion auxiliary device 100 and the distal end of the guiding tube 120 are arranged in the patient's trachea. Then, insert the ETT into the patient's body from the avoiding space 116, and then insert the bronchoscope into the insertion auxiliary device 100, and complete the endotracheal intubation directly under the bronchoscope.

This embodiment relates to an auxiliary device for inserting the insertion auxiliary device 100, which has at least the following advantages:

The insertion auxiliary device 100 provided by this embodiment is arranged with an avoiding space 116, so that the ETT can be inserted into the human body from the outside of the insertion auxiliary device 100, and a bronchoscope is inserted into the human body through the passaged formed by inserting the insertion auxiliary device 100; therefore, the protection of the bronchoscope is realized through the insertion auxiliary device 100, and the obstruction of the sight of the insertion auxiliary device 100 is prevented, avoiding the problem of biting of the bronchoscope. The passage established with the insertion auxiliary device 100 can help realize the rapid exchange of the bronchoscope and other devices, reduce the secretion brought out by the device from the lumen, and lower the risk of infection among medical staff. At the same time, it improves the visualization and ventilation of the surgical procedure, which helps to reduce the difficulty in intubation. Therefore, the air sac at the distal end of the ETT can be placed at an appropriate position under direct vision to replenish itself and block the respiratory tract during the surgery according to the demand, which can avoid the problem of ejection of a large amount of aerosols during tracheotomy, thus lowering the risk of infection for medical staff. In addition, the tongue depressor 130 is provided, which not only holds the tongue when the insertion auxiliary device 100 enters the mouth, but also facilitates the guiding tube 120 to enter the throat of the human body, and further facilitates the bronchoscope to directly enter the trachea through the guiding tube 120, thus further lowering the difficulty in operation.

This embodiment also relates a medical device (not illustrated in the drawing), which comprises an interventional medical device and the said insertion auxiliary device 100. Therefore, it has all the functions of the insertion auxiliary device 100.

The above disclosure describes various embodiments, while the protection scope of this disclosure is not confined to those embodiments. The changes or replacements under the control of any person skilled in the art within the disclosed scope shall be covered in the protection scope of this disclosure. Therefore, the protection scope of this disclosure shall be subject to the protection scope of the claims.

1. An auxiliary device for inserting a medical device, comprising:
a guiding tube configured to receive the medical device; and
a bite part coupled to a proximal end of the guiding tube, the bite part including an insertion cavity in communication with the guiding tube and defined by a first retaining part and a second retaining part;
wherein the first retaining part is configured to contact an upper lip of a patient and the second retaining part is configured to contact a lower lip of a patient; and
wherein the first retaining part, the second retaining part, the upper lip, and the lower lip define an avoiding space configured to receive an additional medical device.

2. The auxiliary device of claim 1, wherein the medical device is a bronchoscope and the additional medical device is an endotracheal tube.

3. The auxiliary device of claim 1, further comprising:
a first toothing part coupled to and extending distally from the first retaining part and configured to contact upper teeth of the patient; and
a second toothing part coupled to and extending distally from the second retaining part and configured to contact lower teeth of the patient.

4. The auxiliary device of claim 1, further comprising:
an oxygen passage extending from a proximal end of the bite part to a distal end of the guiding tube, the oxygen passage positioned outside of the guiding tube.

5. The auxiliary device of claim 4, wherein the oxygen passage is made of elastic material.

6. The auxiliary device of claim 1, wherein the guiding tube is shaped like an arc and includes an inside curve and an outside curve to facilitate insertion of the guiding tube into a trachea of the patient.

7. The auxiliary device of claim 6, further comprising a tongue depressor coupled to a distal end of the guiding tube along the inside curve, the tongue depressor configured to a tongue of the patient to facilitate insertion of the guiding tube into the trachea.

8. An auxiliary device for inserting a medical device, comprising:
a guiding tube configured to receive the medical device; and
a bite part coupled to a proximal end of the guiding tube, the bite part further comprising:
a first toothing part;
a second toothing part positioned opposite the first toothing part;
a first connecting part that couples the first toothing part and the second toothing part; and
a second connecting part that couples the first toothing part and the second toothing part, the second connecting part positioned opposite the first connecting part;
wherein an insertion cavity is defined by the first toothing part, the second toothing part, the first connecting part, and the second connecting part, and wherein the second connecting part is recessed toward the first connecting part and forms a groove that defines an avoiding space configured to receive an additional medical device.

9. The auxiliary device of claim 8, further comprising:
a first retaining part positioned between, and coupled to, the guiding tube and the first toothing part;
a second retaining part positioned between, and coupled to, the guiding tube and the second toothing part;
a first extending part positioned between, and coupled to, the guiding tube and the first connecting part; and
a second extending part positioned between, and coupled to, the second connecting part;
wherein the groove extends through the second extending part and the second extending part further defines the avoiding space.

10. The auxiliary device of claim 9, wherein the first retaining part is configured to contact an upper lip of a patient and the second retaining part is configured to contact a lower lip of a patient.

11. The auxiliary device of claim 8, wherein the medical device is a bronchoscope and the additional medical device is an endotracheal tube.

12. The auxiliary device of claim 8, wherein the guiding tube is shaped like an arc and includes an inside curve and an outside curve to facilitate insertion of the guiding tube into a trachea of a patient.

13. The auxiliary device of claim 12, further comprising an oxygen passage extending along the arc of the guiding tube from a proximal end of the bite part to a distal end of the guiding tube.

14. The auxiliary device of claim 13, wherein the oxygen passage follows the outside curve.

15. An auxiliary device for inserting a medical device, comprising:
a guiding tube configured to receive the medical device, the guiding tube shaped like an arc and including an inside curve and an outside curve; and
a tongue depressor comprising:
a tongue depressing part coupled to the guiding tube at a distal end of the guiding tube and along the inside curve of the guiding tube; and
a support part coupled to a side edge of the tongue depressing part.

16. The auxiliary device of claim 15, wherein the tongue depressing part and the support part form an accommodation space configured to receive a bronchoscope and prevent the bronchoscope from contacting a trachea of a patient.

17. The auxiliary device of claim 16, wherein a wall of the support part positioned opposite the tongue depressing part is a cambered surface.

18. The auxiliary device of claim 17, wherein a distance between the cambered surface and the tongue depressing part decreases as the cambered surface extends toward a distal end of the tongue depressing part.

19. The auxiliary device of claim 15, wherein the tongue depressor and the guiding tube are a single component.

20. The auxiliary device of claim 15, wherein the auxiliary device is made from a transparent material.
